(19) United States
(12) Patent Application Publication
(10) Pub. No.: US 2001/0031261 A1
(43) Pub. Date: Oct. 18, 2001

(54) Use of genetically engineered antibodies to treat multiple myeloma

(76) Inventors: John A. Lust, Rochester, MN (US); Kathleen A. Donovan, Rochester, MN (US)

Correspondence Address: SCHWEGMAN, LUNDBERG, WOESSNER & KLUTH, P.O. BOX 2938, MINNEAPOLIS, MN, 55402, US

(21) Appl. No.: 09/730,374

(22) Filed: Dec. 5, 2000

Related U.S. Application Data
(63) Continuation of application No. PCT/US99/12512, filed on Jun. 4, 1999.
(60) Provisional application No. 60/088,277, filed on Jun. 5, 1998.

(51) Int. Cl. A61K 39/395; C07H 21/04; C12P 21/04; C07K 16/30

(52) U.S. Cl. 424/178.1; 536/23.5; 435/69.7; 530/391.1

(57) ABSTRACT

A fusion polypeptide comprising an antibody or a fragment thereof is provided. The fusion polypeptide is useful in a therapeutic composition to treat multiple myeloma.

### BACKGROUND OF THE INVENTION

Multiple myeloma is a fatal neoplasm characterized by an accumulation of a clone of plasma cells, frequently accompanied by the secretion of Ig chains. Bone marrow invasion by the tumor is associated with anemia, hypogammaglobinemia, and granulocytopenia with concomitant bacterial infections. An abnormal cytokine environment, principally raised IL-6 and IL-1 β levels, often results in increased osteoclasis leading to bone pain, fractures, and hypercalcemia.

Despite aggressive chemotherapy and transplantation, multiple myeloma is a universally fatal plasmaproliferative disorder. As an alternative approach, several workers have proposed immunotherapeutic strategies. In contrast to the nonspecificity of conventional chemotherapy, antibodies and natural ligands can specifically bind to targets of cancer cells. Antibodies that have been linked to a toxin molecule or a radioisotope form an immunotoxin or an immunoconjugate, which has the potential advantage of specifically killing target cells. One such approach for multiple myeloma has targeted the IL-6-signaling system. IL-6 has been suggested to be a major growth factor for myeloma cells functioning in either an autocrine or paracrine fashion. Two murine monoclonals that neutralize IL-6 suppressed the proliferation of myeloma cells in a patient with a leukemic variant of the disease, although the tumor relapsed after 60 days. Similarly, the IL-6R has been investigated as a target for both blocking antibodies (Abs) and IL-6-cytotoxin conjugates.

Although immunotoxins are theoretically highly specific tumor cell killing agents, currently used immunotoxins have several major problems, such as immunogenicity, the limited accessibility of circulating immunotoxins to tumor cells, and side effects, which greatly limit their efficacy for cancer therapy. In particular, neutralizing antibodies to the toxins develop shortly after exposure to the immunotoxins, even with concomitant use of immunosuppressive agents, thus greatly preventing their repeated administration and therapeutic efficacy in patients. The use of humanized murine antibodies has solved, to a great extent, the immunogenicity problem of the targeting moiety, but for the highly immunogenic toxin moiety, the problem still remains. Moreover, another potential problem of immunotoxins is the fact that tumor antigens are not exclusive tumor-specific antigens; some normal human tissues also display the antigens though at a considerably lower level.

Thus, what is needed is a therapeutic molecule that is highly specific for multiple myeloma cells but has low or no immunogenicity.

### SUMMARY OF THE INVENTION

The present invention provides a genetically engineered (recombinant) fusion polypeptide comprising at least a portion of a polypeptide that specifically binds CD38 antigen that is linked to at least a portion of a polypeptide that is a DNA binding protein. Thus, the invention also provides an isolated and purified nucleic acid molecule, e.g., a DNA molecule, sequence or segment, which encodes a fusion polypeptide of the invention. The fusion polypeptide of the invention is useful in the therapeutic compositions of the invention, which are described hereinbelow, which compositions can inhibit or treat conditions characterized by the presence or proliferation of CD38-expressing cells.

CD38 is a cell surface antigen that is known to be expressed in high density on virtually all malignant plasma cells from the majority of myeloma patients. Although CD38 is present on resting natural killer cells, activated T and B cells, and bone marrow precursor cells, it is expressed in highest density on normal plasma cells which often comprise less than 1% of normal bone marrow cells. Early stem cells needed to replenish these populations are CD38 negative.

Preferably, the CD38 binding polypeptide is an antibody, a fragment or a variant thereof, e.g., (Fab′)₂, Fab, Fv, Fd, light chain or heavy chain dimers, chimeric antibodies or single chain antibodies, such as a single chain variable fragment (scFv). More preferably, the CD38 binding polypeptide is a recombinant antibody that is a single chain variable region fragment (scFv), which is optionally humanized. A preferred embodiment of the invention is a fusion polypeptide comprising an immunoglobulin heavy chain from a hybridoma secreting an anti-CD38 antibody linked to an immunoglobulin light chain from the same hybridoma. Preferably the linkage is by a hydrophilic peptide bridge. The fusion polypeptide has affinity and specificity for the CD38 antigen and is internalized by CD38+ cells, e.g., myeloma cells.

The invention further provides a recombinant DNA molecule that encodes a single chain fusion polypeptide. The recombinant DNA molecule comprises: a) a DNA sequence that encodes the Fv region of a light chain of an antibody specific for CD38 and the Fv region of a heavy chain of an antibody specific for CD38, wherein the fusion protein binds to CD38⁺ cells; and b) a DNA sequence that encodes a polypeptide that specifically binds DNA. Therefore, the invention also provides a recombinantly produced single chain fusion polypeptide comprising: a) the Fv region of the light and the heavy chain of a CD38 specific antibody; and b) a DNA binding polypeptide, wherein the Fv region and the DNA binding polypeptide are recombinantly fused to form a single chain polypeptide that specifically binds CD38⁺ cells.

A preferred DNA binding polypeptide of the invention includes, but is not limited to, protamine, histone or polylysine. More preferably, the DNA binding polypeptide is protamine. To link a DNA binding polypeptide gene to a CD38 binding polypeptide gene, polymerase chain reaction (PCR) overlap extension techniques may be employed. In one preferred embodiment, the DNA binding polypeptide is not an antibody, enzyme or a cytotoxic agent.

Also provided is a therapeutic composition which comprises a fusion polypeptide of the invention and a DNA molecule encoding a cytotoxic agent. Thus, the fusion polypeptide functions as a carrier to introduce a therapeutic gene encoding a cytotoxic agent, e.g., toxin genes such as diphtheria toxin-A, lectins, Pseudomonas exotoxin A, Saponaria officinalis SO-6 (Soria, Pharna. Res., 21, 35 (1989)) or ricin; cell suicide genes such as thynidine kinase or nitroreductase; proteins that activate chemotherapeutic genes such as gangcyclovir or mitomycin C; a ribozyme, RNase, or an antisense sequence (e.g., BCL2 sequence); into CD38+ cells such as myeloma cells. This is in contrast to WO 96/16990 which suggests that anti-CD38 antibodies or humanized versions thereof are useful to treat multiple myeloma, as a result of host effector cells lysing cells coated with such antibodies. Preferably, the expression of only a few molecules of the cytotoxic agent encoded by the therapeutic gene are sufficient to kill a cell that expresses that gene. It is preferred that the therapeutic gene is operatively linked to a cell or tissue-specific transcription unit, e.g., a cell or tissue-specific promoter and/or enhancer. Preferred transcription units are those which direct expression in B cells (e.g., transcription units from an Ig heavy gene, Ig kappa gene, Ig lambda gene, BCL-6 gene (Dalla Favera et al., C.S.H. Smp. Quant. Biol., 59, 117 (1994)), CD19 gene, CD20 gene, or CD22 gene (Kerhl et al., Immunol. Today, 15, 432 (1994)), T cells (e.g., transcription units from the IL-4 gene, IL-2 gene, IL-2R gene, T cell receptor gene, IL-5 gene, IL-13 gene, GM-CSF gene and Fas ligand gene (Nagata et al., Prog. Mol. Subcell. Biol., 16, 87 (1996)) or myeloid cells. Myeloid-specific transcription units include, but are not limited to, those disclosed in U.S. Pat. No. 5,502,176, as well as transcription units from the PU.1 gene (Fisher et al., Stem Cells, 16, 25 (1998)), CD11c or CD18 gene (Corbi et al., Leuk. & Lymph., 25, 415 (1997)), IgH enhancer, CSF receptor G, GM and/or G gene (Zhang et al., Cur. Top. Micro. & Immunol., 211, 137 (1996)), or the C/EBP, Runt/PEBP2/CBF or Ets gene (Clarke et al., J. Leuko, Biol., 63, 153 (1998)).

Thus, the invention provides a therapeutic composition which selectively targets CD38 cell surface molecules but has reduced or no immunogenicity as the therapeutic gene, preferably in the form of circular DNA such as plasmid DNA, rather than an immunogenic protein, is introduced to the host mammal. As a result, it may be possible to repeatedly administer the therapeutic composition to a mammal, e.g., myeloma patients, without the development of significant antibody responses, particularly to the cytotoxic agent encoded by the therapeutic gene. Moreover, a therapeutic composition of the invention is useful to kill cells in patients with other CD38+ plasmaproliferative disorders such as primary amyloidosis, monoclonal gammopathy of undetermined significance and acute myeloid leukemia. Preferably, a humanized version of the antibody portion of the fusion polypeptide in the composition is employed for use in humans.

Further provided is a pharmaceutical composition comprising a recombinantly produced single chain fusion polypeptide in a concentration sufficient to inhibit tumor cell growth, together with a pharmaceutically acceptable carrier. The fusion polypeptide comprises: a) a single chain Fv region of an antibody, wherein the Fv region comprises the V_{H} and V_{L} regions of the antibody; and b) a DNA binding polypeptide, wherein the Fv region and the DNA binding polypeptide are recombinantly fused to form a single molecule that specifically binds CD38⁺ cells.

Also provided is a method to inhibit the growth of CD38+ cells, comprising contacting cells with an effective amount of a therapeutic composition of the invention. Preferably, the composition is administered to a mammal in an amount that is effective to inhibit or treat multiple myeloma, primary amyloidosis, monoclonal gammopathy, or acute myeloid leukemia.

The CD38 binding portion of the fusion polypeptide of the invention is also useful as a transfection reagent to introduce any gene of interest into a CD38+ cell in vitro, as hematologic cells are difficult to transfect by other conventional approaches, or in vivo, e.g., in an animal model such as the ARH-77 SCID mouse.

Another embodiment of the invention is a fusion polypeptide of the invention which is linked, e.g., chemically fused, to an cytotoxic agent such as a radioactive isotope (e.g., ¹²⁵I, ¹³¹Cs, ³²p, ⁹⁰Y, ¹⁴C, ³H, and ³⁵S), i.e., it is a radioimmunoconjugate, or to another molecule, e.g., a drug, toxin, cofactor, substrate, inhibitor, magnetic marker, fluorescent marker, chemiluminescent marker, and the like.

### BRIEF DESCRIPTION OF THE FIGURES

FIG. 1 depicts a FACS analysis of the binding of anti-CD38 antibodies to normal and myeloma cells.

FIG. 2 depicts a FACS analysis of the binding of anti-CD38 scFv to CD38+ ARH-9 cells.

FIG. 3 depicts a Western blot analysis showing the internalization of scFv antibodies (expressed from constructs F5-1 and C5-2) in CD38⁺ 8226 myeloma cells. T=total uptake. A=after acid wash.

FIG. 4 shows a schematic diagram of an exemplary fusion polypeptide of the invention. V_{H}=heavy chain variable region; L=linker; V_{L}=light chain variable region; P=protein.

FIG. 5 shows the binding of a therapeutic composition of the invention to a CD38⁺ expressing cell. The circle with two boxes indicates a plasmid molecule encoding diphtheria toxin A.

FIG. 6 depicts the nucleotide sequence (SEQ ID NO:1) encoding a scFv which specifically binds CD38.

### DETAILED DESCRIPTION OF THE INVENTION

Definitions

Abbreviations used for the twenty naturally occurring amino acids, the five naturally occurring nucleic acids and the eleven nucleic acid degeneracies (wobbles) follow conventional usage. In the polypeptide notation, the left-hand direction is the amino terminal direction and the right-hand direction is the carboxy-terminal direction. In the nucleic acid notation, the left-hand direction is the 5′ direction and the right-hand direction is the 3′ direction.

The term “nucleic acid” refers to a deoxyribonucleotide or ribonucleotide polymer in either single- or double-stranded form, and unless otherwise limited, would encompass known analogs of natural nucleotides that can function in a manner similar to naturally occurring nucleotides.

The phrase “nucleic acid encoding” or “nucleic acid sequence encoding” refers to a nucleic acid, i.e., DNA or RNA, which directs the expression of a specific polypeptide, protein or peptide. The nucleic acid sequences include both the DNA strand sequence that is transcribed into RNA and the RNA sequence that is translated into protein. The nucleic acid sequences include both full length nucleic acid sequences as well as shorter sequences derived from the full length sequences. It is understood that a particular nucleic acid sequence includes the degenerate codons of the native sequence or sequences which may be introduced to provide codon preference in a specific host cell. The nucleic acid includes both the sense and antisense strands as either individual single strands or in the duplex form. An isolated “variant” nucleic acid molecule of the invention is a nucleic acid molecule which has at least 80%, preferably at least about 90%, and more preferably at least about 95%, but less than 100%, contiguous nucleotide sequence homology or identity to the nucleotide sequence encoding a fusion polypeptide of the invention or DNA encoding a cytotoxic agent. For example, a variant of a nucleic acid molecule encoding a fusion polypeptide has at least 80%, preferably at least about 90%, and more preferably at least about 95%, but less than 100%, contiguous nucleotide sequence homology or identity to the nucleotide sequence comprising SEQ ID NO:1. Moreover, a variant nucleic acid molecule of the invention may include nucleotide bases not present in the corresponding non-variant nucleic acid molecule, as well as internal deletions relative to the corresponding wild type nucleic acid molecule.

An isolated “variant” of a fusion polypeptide of the invention is a polypeptide which has at least about 50%, preferably at least about 80%, and more preferably at least about 90%, but less than 100%, contiguous amino acid sequence homology or identity to the amino acid sequence encoded by SEQ ID NO: 1. For example, it is preferred that the variant has at least about 10% of the biological activity, e.g., binding to CD38, of the corresponding non-variant polypeptide, such as a polypeptide encoded by SEQ ID NO:1. Conservative amino acid substitutions are preferred--that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. The invention also envisions polypeptide or peptide variants with non-conservative substitutions. Non-conservative substitutions entail exchanging a member of one of the classes described above for another. After the substitutions are introduced, the variants are screened for biological activity.

The terms “isolated” or “substantially purified,” when referring to recombinantly produced polypeptides, or DNA encoding a cytotoxic agent of the invention, means a chemical composition which is essentially free of other cellular components. Such a composition is preferably in a homogeneous state although it can be in either a dry or aqueous solution. Purity and homogeneity are typically determined using analytical chemistry techniques such as polyacrylamide gel electrophoresis or high performance liquid chromatography (for polypeptides), or A₂₆₀/A₂₈₀ ratios (for nucleic acids). A polypeptide which is the predominant species present in a preparation is substantially purified. Generally, a substantially purified or isolated polypeptide or nucleic acid molecule comprises more than 80% of all macromolecular species present in the preparation. Preferably, the polypeptide or nucleic acid molecule is purified to represent greater than 90% of all macromolecular species present. More preferably the polypeptide or nucleic acid molecule is purified to greater than 95%, and most preferably the polypeptide or nucleic acid molecule is purified to essential homogeneity, wherein other macromolecular species are not detected by conventional techniques.

As used herein “ligands” or “binding moieties” are molecules capable of reacting with or otherwise recognizing and specifically binding a “target” molecule. Ligands and their respective target molecules represent paired species. Typical paired species include, but are not limited to, enzyme/substrate, receptor/agonist, antibody/antigen, and lectin/carbohydrate. The binding between a ligand and its target may be mediated by covalent or non-covalent interactions or a combination of covalent and non-covalent interactions. When the interaction of the two species produces a non-covalently bound complex, the binding which occurs is typically electrostatic, hydrogen-bonding, or the result of hydrophilic/lipophilic interactions. Accordingly, “specific binding” occurs between a ligand and its target molecule where there is interaction between the two which produces a bound complex having the characteristics of an antibody/antigen or enzyme/substrate interaction. Specifically, examples of ligands include, but are not limited to antibodies, lymphokines, cytokines, receptor proteins such as CD38, solubilized receptor proteins such as soluble CD38, hormones, growth factors, and the like which specifically bind desired target cells.

The phrase “binding specificity,” or “specifically immunoreactive with,” refers to a binding reaction which is determinative of the presence of a protein in the presence of a heterogeneous population of proteins and other biologics. Thus, under particular conditions, the fusion polypeptides of the invention bind to a particular protein, i.e., CD38, and do not bind in a significant amount to other proteins or carbohydrates present in the sample. Specific binding to CD38 under such conditions may require an antibody that is selected for its specificity for a particular protein or carbohydrate. A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. See Harlow and Lane (1988) Antibodies, a Laboratory Manual, Cold Spring Harbor Publications, New York, for a description of immunoassay formats and conditions that can be used to determine specific immunoreactivity.

The terms “recombinant DNA,” “recombinant nucleic acid” or “recombinantly produced DNA” refer to DNA which has been isolated from its native or endogenous source and modified either chemically or enzymatically by adding, deleting or altering naturally occurring flanking or internal nucleotides. Flanking nucleotides are those nucleotides which are either upstream or downstream from the described sequence or sub-sequence of nucleotides, while internal nucleotides are those nucleotides which occur within the described sequence or sub-sequence.

The term “labeled antibody” as used herein refers to an antibody bound to a label such that detection of the presence of the label (e.g., as bound to a biological sample) indicates the presence of the antibody.

A “cytotoxic agent” refers to a molecule that when contacted with a cell brings about the death of that cell. A cytotoxic agent of the invention includes, but is not limited to, agents such as bacterial or plant toxins, drugs, e.g., cyclophosphamide (CTX; cytoxan), chlorambucil (CHL; leukeran), cisplatin (CisP; CDDP; platinol), busulfan (myleran), melphalan, carmustine (BCNU), streptozotocin, triethylenemelamine (TEM), mitomycin C, and other alkylating agents; methotrexate (MTX), etoposide (VP-16; vepesid), 6-mercaptopurine (6MP), 6-thioguanine (6TG), cytarabine (Ara-C), 5-fluorouracil (5FU), dacarbazine (DTIC), 2-chlorodeoxyadenosine (2-CdA), and other antimetabolites; antibiotics including actinomycin D, doxorubicin (DXR; adriamycin), daunorubicin (daunomycin), bleomycin, mithramycin as well as other antibiotics; alkaloids such as vincristin (VCR), vinblastine, and the like; as well as other anti-cancer agents including the cytostatic agents glucocorticoids such as dexamethasone (DEX; decadron) and corticosteroids such as prednisone, nucleotide enzyme inhibitors such as hydroxyurea, and the like. The synthesis and formulation of the above anti-cancer drugs is well known, is described in a variety of sources, and therefore will not be repeated here. Exemplary sources for synthesis and formulations of anti-cancer drugs include Physician's Desk Reference, Barnhart, eds., Medical Economics Company, Inc., Oradell, New Jersey, 1992, Merck Index, 11th Edition, Merck & Co., 1989.

When a DNA molecule encoding a cytotoxic agent is present in a therapeutic composition of the invention, the DNA preferably encodes a polypeptide that is a bacterial or plant toxin. These polypeptides include, but are not limited to, polypeptides such as native or modified Pseudomonas exotoxin (PE), diphtheria toxin (DT), ricin, abrin, gelonin, momordin II, bacterial RIPs such as shiga and shiga-like toxin a-chains, luffin [see Islam et al., Agricultural Biological Chem., 54(5):1343-1345 (199_)], atrichosanthin [see Chow et al., J. Biol. Chem., 265:8670-8674 (1990))], momordin I [see Ho et al., BBA, 1088:311-314 (1991)], Mirabilis anti-viral protein [see Habuka et al., J. Biol. Chem., 264(12):6629-6637 (1989)], pokeweed antiviral protein [see Kung et al., Agric. Biol. Chem., 54(12):3301-3318 (1990)], byodin 2 (U.S. Pat. No. 5,597,569), gaporin [see Benatti et al., Eur. J. Biochem., 183:465-470 (1989)], as well as genetically engineered variants thereof. Native PE and DT are highly toxic compounds that typically bring about death through liver toxicity. Preferably, PE and DT are modified into a form that removes the native targeting component of the toxin, e.g., domain Ia of PE and the B chain of DT. One of skill in the art will appreciate that the invention is not limited to a particular cytotoxic agent.

The term “Pseudomonas exotoxin” (PE) as used herein refers to a full-length native (naturally occurring) PE or a PE that has been modified. Such modifications may include, but are not limited to, elimination of domain Ia, various amino acid deletions in domains II and III, single amino acid substitutions (e.g., replacing Lys with Gln at positions 590 and 606), and the addition of one or more sequences at the carboxyl terminus. See Siegall et al., J. Biol. Chem., 264: 14256-14261 (1989). Thus, for example, PE38 refers to a truncated Pseudomonas exotoxin composed of amino acids 253-364 and 381-613. The native C-tenninus of PE, REDLK (residues 609-613), may be replaced with the sequence KDEL, REDL, and Lys-590 and Lys-606 may be each mutated to Gln.

The term “Diphtheria toxin” (DT) as used herein refers to full length native DT or to a DT that has been modified. Modifications typically include removal of the targeting domain in the B chain and, more specifically, involve truncations of the carboxyl region of the B chain.

I. CD38 Binding Moieties

Preferred CD38 binding moieties are polypeptides or compounds identified as having binding affinity to CD38. More preferred CD38 binding moieties are anti-CD38 antibodies (naturally occurring or recombinant, from any source), e.g., THB7, AT/315, and fragments thereof

As used herein, the term “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies may exist as intact immunoglobulins, or as modifications in a variety of forms including, for example, FabFc₂, Fab, Fv, Fd, (Fab′)₂, an Fv fragment containing only the light and heavy chain variable regions, a Fab or (Fab)′₂ fragment containing the variable regions and parts of the constant regions, a single-chain antibody (Bird et al., Science, 242: 424-426 (1988); Huston et al., Proc. Natl. Acad. Sci. USA, 85: 5879-5883 (1988) both incorporated by reference herein), CDR-grafted antibodies and the like. The heavy and light chain of a Fv may be derived from the same antibody or different antibodies thereby producing a chimeric Fv region. The antibody may be of animal (especially mouse or rat) or human origin or may be chimeric (Morrison et al., Proc. Natl. Acad. Sci. USA, 81, 6851-6855 (1984) both incorporated by reference herein) or humanized (Jones et al., Nature, 321, 522-525 (1986), and published UK patent application #8707252, both incorporated by reference herein). As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies of the present invention can be readily made.

The CD38-binding antibodies may be Fv regions comprising a variable light (V_{L}) and a variable heavy (V_{H}) chain. The light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

A preferred embodiment of the invention is a fusion polypeptide comprising a recombinantly produced antibody comprising a V_{H} and C_{H}, or a portion thereof, joined to a DNA binding polypeptide. The fusion polypeptide and an antibody comprising V_{L} and C_{L}, or a portion thereof, together form a recombinant antibody useful to direct preselected DNA molecules, either linear or circular, to a cell or tissue bearing the preselected target molecule.

Another preferred embodiment of the invention is a recombinantly produced single chain scFv antibody, preferably a humanized scFv. In particular, this invention provides for recombinant single chain antibodies that are joined to a DNA binding polypeptide and, because of their ability to specifically bind to DNA, these antibodies are useful as targeting moieties which serve to direct DNA which is bound to DNA binding polypeptide to a cell or tissue bearing the preselected target molecule, i.e., CD38.

The recombinant single chain antibodies of the present invention may be fused to, or otherwise bound to the DNA binding polypeptide and, optionally to a radionuclide or other molecule having a specified activity by any method known and available to those in the art. The two components may be chemically bonded together by any of a variety of well-known chemical procedures. For example, the linkage may be by way of heterobifunctional cross-linkers, e.g., SPDP, carbodiimide, glutaraldehyde, or the like. Production of various immunotoxins, as well as chemical conjugation methods, are well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982); Waldmann, Science, 252: 1657 (1991); Vitetta et al., 1987, Science, 238:1098; Pastan et al., 1986; Cell, 47:641; and Thorpe et al., 1987, Cancer Res., 47:5924, which are incorporated by reference herein. These methods generally conjugate the DNA binding polypeptide and the antibody by means of cross-linking agents that introduce a disulfide bond between the two polypeptides. Immunotoxins which have been prepared with nonreducible linkages have been shown to be consistently less cytotoxic than similar toxins cross-linked by disulfide bonds.

Other preferred reagents are 2-iminothiolane hydrochloride (2IT), sodium S-4-succinimidyloxycarbonyl-α-methyl benzyl thiosulfate (SMBT) and 2IT or succinimidyloxy carbonyl-α-methyl-α(2-pyridyldithio)-toluene and 2IT. Each group of reagents introduces a disulfide bond between the DNA binding polypeptide and the antibody which is reducible, but the bond is also resistant to breakdown providing stability of the conjugate in vitro and in vivo. Upon internalization into lysosomes or endosomes by the target cell, the bond is reduced. For example, to use the recombinant PE molecules with an antibody, a form of the PE molecule with cysteine at amino acid position 287 is preferred to couple the toxin to the antibody or other ligand through the thiol moiety of cysteine.

In a preferred embodiment, the CD38 binding moiety may also be fused to a DNA binding polypeptide by recombinant means such as through the use of recombinant DNA techniques to produce a nucleic acid which encodes both the antibody and the DNA binding polypeptide and expressing the recombinant DNA sequence in a host cell, such as a eukaryotic, e.g., mammalian such as CHO or COS cells, or prokaryotic, e.g., E. coli, host. The DNA encoding the fusion polypeptide may be cloned in cDNA or in genomic form by any cloning procedure known to those skilled in the art. See for example Sambrook et al., Molecular Cloning: a Laboratory Manual, Cold Spring Harbor Laboratory, (1989), which is herein incorporated by reference.

Fusion or conjugation of the fusion polypeptide of the invention to various labels produces a highly specific detectable marker that may be used to detect the presence or absence of cells or tissues having the particular molecule to which the antibody binds, i.e., CD38. Alternatively, the fusion polypeptide may be chemically conjugated or fused to a molecule that is another specific binding moiety, e.g., a ligand. In this form, the fusion polypeptide can act as a highly specific bifunctional linker. This linker may act to bind and enhance the interaction between cells or cellular components to which the fusion protein binds.

A. Preparation of Genes Encoding Antibodies or Fragments Thereof

Genes encoding antibodies, both light and heavy chain genes or portions thereof, e.g., single chain Fv regions, may be cloned from a hybridoma cell line. The may all be cloned using the same general strategy. Typically, for example, poly(A) +RNA extracted from the hybridoma cells is reverse transcribed using random hexamers as primers. For Fv regions, the V_{H} and V_{L} domains are amplified separately by two polymerase chain reactions (PCR). Heavy chain sequences may be amplified using 5′ end primers which are designed according to the amino-terminal protein sequences of the anti-CD38 heavy chains respectively and 3′ end primers according to consensus immunoglobulin constant region sequences (Kabat et al., Sequences of Proteins of Immunological Interest. 5th edition. U.S. Department of Health and Human Services, Public Health Service, National Institutes of Health, Bethesda, Md. (1991) incorporated by reference). Light chain Fv regions are amplified using 5′ end primers designed according to the amino-terminal protein sequences of anti-CD38 light chains and in combination with the primer C-kappa. One of skill in the art would recognize that many suitable primers may be employed to obtain Fv regions.

The PCR products are subcloned into a suitable cloning vector. Clones containing the correct size insert by DNA restriction are identified. The nucleotide sequence of the heavy or light chain coding regions may then be determined from double stranded plasmid DNA using sequencing primers adjacent to the cloning site. Commercially available kits (e.g., the Sequenase™ kit, United States Biochemical Corp., Cleveland, Ohio, USA) may be used to facilitate sequencing the DNA.

Thus, DNA encoding the Fv regions may be prepared by any suitable method, including, for example, amplification techniques such as ligase chain reaction (LCR) (see Wu and Wallace, Genomics, 4: 560 (1989), Landegren, et al., Science, 241: 1077 (1988) and Barringer, et al., Gene, 89: 117 (1990)), transcription amplification (see Kwoh, et al., Proc. Natl. Acad. Sci. USA, 86: 1173 (1989)), and self-sustained sequence replication (see Guatelli, et al., Proc. Natl. Acad. Sci. USA, 87: 1874 (1990)), cloning and restriction of appropriate sequences or direct chemical synthesis by methods such as the phosphotriester method of Narang et al., Meth. Enzymol. 68: 90-99 (1979); the phosphodiester method of Brown et al., Meth Enzymol. 68: 109-151 (1979); the diethylphosphoramidite method of Beaucage et al., Tetra. Lett., 22: 1859-1862 (1981); and the solid support method of U.S. Pat. No. 4,458,066, all such references in this paragraph incorporated by reference herein.

Chemical synthesis produces a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, sub-sequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, using techniques well known to those of skill in the art. In one embodiment, heavy and light chain regions are connected by a flexible peptide linker (e.g., (Gly₄Ser)₃) which starts at the carboxyl end of the heavy chain Fv domain and ends at the amino terminus of the light chain Fv domain. The entire sequence encodes the Fv domain in the form of a single-chain antigen binding protein.

B. Preparation of Antibody Fusion Polypeptides

Once a DNA sequence has been identified that encodes a CD38 binding polypeptide which, when expressed, shows specific binding activity, fusion polypeptides comprising that region may be prepared by methods known to one of skill in the art. Thus, the gene encoding the Fv region is fused to a gene encoding a DNA binding moiety, preferably a moiety which is a polypeptide. Optionally, the Fv gene and DNA binding moiety gene are linked to a segment encoding a peptide connector. The resultant fusion polypeptide may also optionally be linked to another molecule (e.g., a radionuclide). The peptide connector may be present simply to provide space between the CD38 targeting moiety and the DNA binding moiety or to facilitate mobility between these regions to enable them to each attain their optimum conformation. The DNA sequence comprising the connector may also provide sequences (such as primer sites or restriction sites) to facilitate cloning or may preserve the reading frame between the sequence encoding the targeting moiety and the sequence encoding the DNA binding protein. The design of such connector peptides is well known to those of skill in the art.

Methods of producing and isolating polypeptides are well known to those of skill in the art. Thus, for example, Chaudhary et al., Nature, 339: 394-97 (1989); Batra et al., J. Biol Chem., 265: 15198-15202 (1990); Batra et al., Proc. Natl. Acad. Sci. USA, 87: 8545-8549 (1989); Chaudhary et al., Proc. Natl. Acad. Sci. USA, 81: 1066-1070 (1990), all incorporated by reference, describe the preparation of various single chain antibody polypeptides.

Generally producing fusion polypeptides involves separately preparing the Fv light and heavy chains and DNA encoding any other protein to which they are fused and recombining the DNA sequences in a plasmid or other vector to form a construct encoding the particular desired fusion polypeptide. However, a simpler approach involves inserting the DNA encoding the particular Fv region into a construct already encoding the desired second polypeptide. The DNA sequence encoding the Fv region is inserted into the construct using techniques well known to those of skill in the art.

For each Fv, the V_{H} and V_{L} sequences are PCR amplified using the heavy chain and light chain in their respective plasmids as templates. The amplification primers are designed to have at their ends sequences that are complementary to the translation initiation, peptide linker and connector which are common to the single-chain Fv-DNA binding protein expression vectors. The PCR products are purified and annealed to a uracil-containing single stranded DNA corresponding to the pUC17 DNA prepared by rescue of pUC17 with a helper phage. The annealed PCR products are extended using the single stranded DNA as a template (see, for example, MUTAGENE® mutagenesis protocol, Biorad, Hercules, Calif., USA). The intact DNA may be used to transform cells and express the fusion polypeptide.

Alternatively, two constructs may be prepared, one encoding a V_{H}C_{H}, or a portion thereof, DNA binding polypeptide fusion and the other encoding V_{K}C_{K}, or a portion thereof. A host cell transfected with both constructs expresses a recombinant antibody fusion polypeptide.

C. The Preparation of DNA Encoding Variable Domain Shuffled Fusion Polypeptides

Chimeric Fv regions containing variable heavy and light chain domains from different, albeit related, antibodies may show significantly greater stability in vitro and in vivo than Fv regions where both the heavy and light domain are derived from the same antibody. Nucleic acids encoding chimeric Fv regions are easily prepared using the techniques described above. The V_{H} and V_{L} sequences are PCR amplified using the heavy chain and light chain in their respective plasmids as templates as described. However, instead of using the V_{H} and V_{L} DNA from the same antibody, the V_{H} and V_{L} DNAs are selected from different antibodies. The DNAs are annealed to a uracil-containing single stranded DNA corresponding to the pUC17 DNA and the synthesis of the fusion protein DNA is completed as described above.

One of skill will appreciate that it is possible to eliminate the DNA binding moiety and express the CD38 binding moiety, e.g., chimeric or single antibody Fv regions, alone. The CD38 binding moieties may be used in various chemical conjugates for example, either directly with toxins or other therapeutic agents, with carriers for therapeutic agents such as liposomes, or with various labels and markers such as fluorescent labels.

D. Humanized Antibodies

Because monoclonal antibodies are non-human antibodies, repeated administration of either labeled antibodies or fusion polypeptides including these antibodies as targeting moieties can result in the formation of antibodies to the administered non-human antibodies (Parren et al., Hum Antibod. Hybridomas, 3: 137-145 (1992)). This immune response may preclude long term treatment in some cases. Therefore it is desirable to produce less immunogenic molecules.

As a first step in making less immunogenic molecules the Fv portion of the non-human, e.g., murine, antibody is humanized so that it may then be used to replace the Fv portion of the murine antibody in the fusion proteins of the present invention. Humanized antibodies are non-human antibodies in which some or all of the amino acid residues are replaced with the corresponding amino acid residue found in a similar human antibody. Humanization thereby reduces the antigenic potential of the antibody.

Antibody variable domains have been humanized by various methods, such as CDR grafting (Riechmann et al., Nature, 332: 323-327 (1988)), replacement of exposed residues (Padlan, Mol. Immunol., 28: 489-498 (1991)) and variable domain resurfacing (Roguska et al., Proc. Natl. Acad. Sci. USA, 91: 969-973 (1994)), all incorporated by reference. The minimalistic approach of resurfacing is particularly suitable for antibody variable domains which require preservation of some mouse framework residues to maintain maximal antigen binding affinity. However, the straightforward CDR grafting approach has also been successfully used for the humanization of several antibodies either without preserving any of the mouse framework residues (Jones et al., Nature, 321: 522-525 (1986) and Verhoeyen et al., Science, 239: 1534-1536 (1988)) or with the preservation of just one or two mouse residues (Riechmann et al., Nature, 332: 323-327 (1988); Queen et al., Proc. Natl. Acad. Sci. USA, 86: 10029-10033 (1989)), all incorporated by reference.

To improve the antibodies of the invention, for therapeutic applications, the Fv portion is humanized by a method referred to as “framework exchange.” In this approach, framework residues are identified that differ from human framework residues in highly homologous human V_{H} or V_{L} donors. These differing framework residues are then simultaneously mutated to human residues. The mutations are introduced onto a single-stranded DNA template prepared from a single-chain cassette which may be expressed in E. coli and allows the rapid purification and analysis of the resulting humanized variants.

This approach combines, yet deviates from the principles of CDR grafting or from the replacement of exposed residues, as some residues that are not normally exposed are humanized, while some other residues that are normally exposed are not humanized. Decisions to preserve certain mouse residues are based on knowledge regarding the effect of mutating these particular residues on the binding affinity of the Fv fragment, or on the possible interactions of these residues with other Fv residues observed in a structural model. For example, Ab Fv regions may be modeled using the AbM package (Oxford Molecular Ltd., Oxford, UK), which implements a combined algorithm of homology and conformational generation techniques. Steepest descents energy minimization and constant temperature simulations (5000 s at 30 K) of solvated models were performed using SYBYL (Tripos, St. Louis, Mo.).

More specifically, humanization is accomplished by aligning the variable domains of the heavy and light chains with the best human homolog identified in sequence databases such as GENBANK or SWISS-PROT using the standard sequence comparison software as described above. Sequence analysis and comparison to a structural model based on the crystal structure of the variable domains of a monoclonal antibody (Queen et al., Proc. Natl. Acad. Sci. USA, 86: 10029-10033 (1989) and Satow et al., J. Mol. Biol., 190: 593-604 (1986)); Protein Data bank Entry IMCP) allows identification of the framework residues that differ between the mouse antibody and its human counterpart.

V_{H} and V_{L} gene segments (e.g., in plasmid pUC17) encoding wild type anti-CD38 may be independently humanized by site specific mutagenesis. One of skill in the art will appreciate that once the Fv region has been cloned and sequenced, alteration of various residues by site specific mutagenesis is routine using standard techniques well known to those of skill in the art (Kunkel, Proc. Natl. Acad. Sci. USA, 82: 488-492 (1985)).

The techniques for cloning and expressing DNA sequences encoding the amino acid sequences corresponding to a single chain derivative of a parental antibody are well known to the skilled artisan, as discussed above.

III. Expression Cassettes and Recombin ant Polypeptides

Methods which are well known to those skilled in the art can be used to construct expression vectors containing a coding sequence and appropriate transcriptional/translational control signals. These methods include in vitro recombinant DNA techniques, synthetic techniques and in vivo recombination/genetic techniques. See, for example, the techniques described in Sambrook et al., 1989, Molecular Cloning: A Laboratory Manual, 2nd Ed., Cold Spring Harbor Laboratory, New York.

A variety of host-expression systems can be utilized to express the coding sequence. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence.

Depending on the host/vector system utilized, any of a number of suitable transcription and translation elements including constitutive and inducible promoters, transcription enhancer elements, transcription terminators, etc., can be used in the expression vector (see, e.g., Bitter et al., 1987, Methods in Enzymol., 153:516-544; WO 97/11761 and WO 96/06167). For example, when cloning in bacterial systems, inducible promoters such as pL of bacteriophage λ; plac, ptrup, ptac (ptrp-lac hybrid promoter) and the like may be used. Promoters produced by recombinant DNA or synthetic techniques can also be used to provide for controlled and high level transcription of the inserted coding sequence.

A further embodiment includes an expression vector which contains a chimeric gene encoding the Fd of an anti-CD38 antibody fused to the human protamine polypeptide in one expression cassette and the anti-CD38 kappa chain encoding gene in another expression cassette. Alternatively, each expression cassette may be on a separate expression vector, e.g., a plasmid.

The recombinant Fv regions and fusion proteins incorporating these antibody regions may be expressed in a variety of host cells, including E. coli, other bacterial hosts, yeast, and various higher eukaryotic cells such as the COS, CHO and HeLa cells lines and myeloma cell lines. a particularly preferred host is E. coli. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences include a promoter and preferably an enhancer derived from immunoglobulin genes, SV40, cytomegalovirus, etc., and a polyadenylation sequence, and may include splice donor and acceptor sequences.

The expression vectors of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Once expressed, the recombinant fusion polypeptide can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification, Academic Press, Inc., New York (1990)). Substantially pure compositions of at least about 90 to 95% homogeneity are preferred, and 98 to 99% or more homogeneity are most preferred for pharmaceutical uses. Once purified, partially or to homogeneity as desired, the polypeptides may then be used therapeutically.

One of skill in the art would recognize that after chemical synthesis, biological expression, or purification, a fusion polypeptide may possess a conformation substantially different than the native antibody. In this case, it may be necessary to denature and reduce the polypeptide and then to cause the polypeptide to re-fold into the preferred conformation. Methods of reducing and denaturing the polypeptide and inducing re-folding are well known to those of skill in the art. (See, Debinski et al., J. Biol. Chem, 268: 14065-14070 (1993); Kreitman and Pastan, Bioconjug. Chem., 4: 581-585 (1993); and Buchner, et al., Anal. Biochem., 205: 263-270 (1992) which are incorporated herein by reference.) Debinski et al., for example, describe the denaturation and reduction of inclusion body proteins in guanidine-DTE. The polypeptide is then refolded in a redox buffer containing oxidized glutathione and L-arginine.

One of skill would recognize that modifications can be made to the fusion polypeptides without diminishing their biological activity. Some modifications may be made to facilitate the cloning, expression, or incorporation of the antibody portion of the fusion polypeptide into the fusion polypeptide. Such modifications are well known to those of skill in the art and include, for example, a methionine added at the amino terminus to provide an initiation site, or additional amino acids placed on either terminus to create conveniently located restriction sites or termination codons.

One of skill will recognize that other modifications may be made. Thus, for example, amino acid substitutions may be made that increase specificity or binding affinity of the fusion polypeptide. Alternatively, non-essential regions of the molecule may be shortened or eliminated entirely. Thus, where there are regions of the molecule that are not themselves involved in the activity of the molecule, they may be eliminated or replaced with shorter segments that serve to maintain the correct spatial relationships between the active components of the molecule. Alternatively more flexible segments may be placed in interdomain regions which then can facilitate folding or production of the molecule (Brinkmann, et al., Proc. Natl. Acad. Sci. USA, 89: 3075-3079 (1992).

Binding activity against CD38 expressing cells is detected by enzyme-linked immunosorbent assay (ELISA) using the culture medium from cells transfected with a vector having DNA encoding a fusion polypeptide sc, while no binding activity is observed in the medium of vector-alone-transformed cells. The secreted fusion polypeptide is purified from serum-free culture medium by using an affinity column coupled with anti-human Ig, e.g., IgG kappa chain, monoclonal antibody. The fusion polypeptide bound to the column is eluted with 100 mM glycine (pH 2.4), concentrated and analyzed by sodium dodecyl sulfate-polyacrylamide gel electrophoresis (SDS-PAGE) under nonreducing and reducing conditions.

The DNA binding activity of the therapeutic composition of the invention is examined by a gel-shift assay. When increasing amounts of the fusion polypeptide are mixed with the radiolabeled DNA fragments or whole plasmid DNA, decreasing amounts of DNA fragments or whole plasmid DNA migrate into the agarose gels and the DNA entering the agarose gels migrates more slowly. In contrast, the DNA incubated with the antibody portion of the fusion polypeptide shows no significant change of its mobility in the agarose gels. The binding activity of the fusion polypeptide to CD38 on the cell surface after it is coupled with DNA is further examined by fluorescence activated cell sorting (FACS). The CD38⁺ cells incubated with either the fusion polypeptide or the fusion polypeptide-DNA complex show positive staining. The cells directly incubated with fluorescein isothiocyanate (FITC)-conjugated antibody also show negative staining.

III. Pharmaceutical Compositions, Dosages, and Routes of Administration

The fusion polypeptides and pharmaceutical compositions of the invention are useful for parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the fusion polypeptides and pharmaceutical compositions of the invention, when administered orally, must be protected from digestion. This is typically accomplished either by complexing the polypeptide and/or DNA with a composition to render them resistant to acidic and enzymatic hydrolysis or by packaging the polypeptide and/or DNA in an appropriately resistant carrier such as a liposome. Means of protecting polypeptides and/or DNA from digestion are well known in the art.

The fusion polypeptides and pharmaceutical compositions of the invention are particularly useful for parenteral administration, such as intravenous administration or administration into a body cavity or lumen of an organ. The compositions for administration will commonly comprise a solution of the fusion polypeptide and/or DNA dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion polypeptide and/or DNA in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.01 to about 100, preferably about 0.1 to about 10, mg per patient per day. Preferably, the composition comprises at least about a 1:1, more preferably about a 1:2, and even more preferably a 1:5, ratio of DNA molecule: fusion polypeptide molecule. Dosages from about 0.1 mg, up to about 1000 mg, per patient per day may be used, particularly when administered to a secluded site and not into the blood stream, such as into a body cavity or into a lumen of an organ. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing the fusion polypeptides or a cocktail thereof (i.e., with other proteins) can be administered for therapeutic treatments. In therapeutic applications, compositions are administered to a patient suffering from a disease, in an amount sufficient to cure or at least partially arrest the disease and its complications. An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the polypeptides and DNA of the invention to effectively treat the patient.

The invention should have therapeutic value in any disease or disorder associated with abnormal (high) expression or abnormal triggering of CD38. For example, it is believed that CD38 plays a role in many basic regulatory processes in immune function. For example, in controlling B cell lymphomas, autoimmune situations, B cell specific proliferative abnormalities (e.g., leukemias), or hypersensitivity responses. Moreover, the fusion polypeptide and compositions of the invention may also have use in systemic lupus erythematosus, myasthenia gravis, non-Hodgkin's lymphoma, rheumatoid arthritis, and non-antibody mediated autoimmune disorders.

The therapeutic composition of the invention can be combined for therapeutic use with additional active ingredients, e.g., in conventional pharmaceutically acceptable carriers or diluents, along with physiologically innocuous stabilizers and excipients; see Berkow (Ed.), The Merck Manual, Merck, Rahway, N.J. These combinations can be filtered sterile and placed into dosage forms as by lyophilization in dosage vials or storage in stabilized aqueous preparations.

The quantities of reagents necessary for effective therapy depend upon many different factors, including means of administration, target site, physiological state of the patient, and other medicaments administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman et al. (Eds.), (1990) Goodman and Gilman's: The Pharmacological Basis of Theraputics, 8th ed., Pergamon Press, Tarrytown, N.Y., and in Remington's Pharmaceutical Sciences, 17th ed. (1990), Mack Publishing Co., Easton, Pa. Methods for administration are discussed therein and below, e.g., for oral, intravenous, intraperitoneal, or intramuscular administration, transdermal diffusion, and others. Pharmaceutically acceptable carriers may include water, saline, buffers, and other compounds described, e.g., in The Merck Index, Merck & Co., Rahway, N.J. See also (e.g.) Avis et al. (Eds.), (1993) Pharmaceutical Dosage Forms: Parenteral Medications,Dekker, New York, and Leiberman et al. (Eds.), (1990) Pharmaceutical Dosage Forms: Disperse Systems, Dekker, New York. Slow-release formulations or slow-release apparatus may be utilized for continuous administration.

Therapeutic formulations may be administered in any conventional dosage formulation. Whereas it is possible for the active ingredient to be administered alone, it is preferable to present it as a pharmaceutical formulation. Formulations comprise at least one active ingredient, together with one or more acceptable carriers therefor. Each carrier must be both pharmaceutically and physiologically acceptable in the sense of being compatible with the other ingredients and not injurious to the patient. Formulations include those suitable for oral, rectal, nasal, or parenteral administration (including subcutaneous, intramuscular, intravenous and intradermal administration). The formulations may conveniently be presented in unit dosage form and may be prepared by any methods well known in the art of pharmacy: e.g., Gilman et al. (Eds.), (1990) Goodman and Gilman's: The Pharmacological Basis of Therapeutics, 8th ed., Pergamon Press; and Remington's Pharmaceutical Sciences, 17th ed. (1990), Mack Publishing Co., Easton, Pa. Further, the invention may be combined with or used in association with other chemotherapeutic or chemopreventive agents.

In particular, the therapeutic compositions of the present invention may be formulated into either an injectable or topical preparation. Parenteral formulations are known and are suitable for use in the invention, preferably for intramuscular or intravenous administration. The formulations containing therapeutically effective amounts of the fusion polypeptide and/or DNA are either sterile liquid solutions, liquid suspensions, or lyophilized versions, and optionally contain stabilizers or excipients. Lyophilized compositions are reconstituted with suitable diluents, e.g., water for injection, saline, 0.3% glycine and the like, at a level of about from 0.01 mg/kg of host body weight to 20 mg/kg. Typically, the pharmaceutical compositions of the present invention are administered in a therapeutically effective dose in a range of from about 0.01 mg/kg to about 5 mg/kg of the patient. A preferred, therapeutically effective dose of the pharmaceutical composition of the invention is in a range of from about 0.01 mg/kg to about 0.5 mg/kg body weight of the patient administered over several days to two weeks by daily intravenous infusion, each given over a one-hour period, in a sequential patient dose-escalation regimen.

Therapeutic compositions according to the invention may be formulated into topical preparations for local therapy by including a therapeutically effective concentration of the fusion polypeptide and/or DNA in a dermatological vehicle. The amount to be administered, and the concentration in the topical formulations, depend upon the vehicle selected, the clinical condition of the patient, the systemic toxicity and the stability of the formulation. Thus, a physician knows to employ the appropriate preparation containing the appropriate concentration of therapeutic agents in the formulation, as well as the appropriate amount of formulation to administered depending upon clinical experience with the patient in question or with similar patients. The concentration of therapeutic compositions for topical formulations is in the range of greater than from about 0.1 mg/ml to about 25 mg/ml. Typically, the concentration of the agents in the composition for topical formulations is in the range of greater than from about 1 mg/ml to about 200 mg/ml. Solid dispersions of the compositions according to the invention, as well as solubilized preparations, may be used. Thus, the precise concentration to be used in the vehicle is subject to modest experimental manipulation in order to optimize the therapeutic response. For example, greater than about 10 mg fusion polypeptide/100 mg DNA encoding a cytotoxic agent/100 grams of vehicle may be useful with 1% w/w hydrogel vehicles. Suitable vehicles, in addition to gels, are oil-in-water or water-in-oil emulsions using mineral oils, petroleum and the like.

Therapeutic compositions according to the invention may be optionally administered topically by the use of a transdermal therapeutic system [Barry, Dermatological Formulations, p. 181 (1983) and literature cited therein]. While such topical delivery systems may be designed for transdermal administration of low molecular weight drugs, they are capable of percutaneous delivery. Further, such systems may be readily adapted to administration of therapeutic polypeptides and/or DNA by appropriate selection of the rate-controlling microporous membrane.

Topical preparations of the therapeutic composition either for systemic or local delivery may be employed and may contain excipients as described above for parenteral administration and other excipients used in a topical preparation such as cosolvents, surfactants, oils, humectants, emollients, preservatives, stabilizers and antioxidants. Pharmacologically acceptable buffers may be used, e.g., Tris or phosphate buffers. The topical formulations may also optionally include one or more agents variously termed enhancers, surfactants, accelerants, adsorption promoters or penetration enhancers, such as an agent for enhancing percutaneous penetration of the therapeutic composition or other agents. Such agents should desirably possess some or all of the following features as would be known to the ordinarily skilled artisan: pharmacological inertness, non-promotive of body fluid or electrolyte loss, compatible with the therapeutic composition (non-inactivating), and capable of formulation into creams, gels or other topical delivery systems as desired.

The therapeutic composition according to the present invention may also be administered by aerosol to achieve localized delivery to the lungs. This is accomplished by preparing an aqueous aerosol, liposomal preparation or solid particles containing the therapeutic composition. Ordinarily, an aqueous aerosol is made by formulating an aqueous solution or suspension of the therapeutic composition together with conventional pharmaceutically acceptable carriers and stabilizers. The carriers and stabilizers vary depending upon the requirements for the particular composition, but typically include: nonionic surfactants (Tweens, Pluronics, or polyethylene glycol); innocuous proteins like serum albumin, sorbitan esters, oleic acid, lecithin; amino acids such as glycine; and buffers, salts, sugars or sugar alcohols. The formulations may also include mucolytic agents as well as bronchodilating agents. The formulations are sterile. Aerosols generally are prepared from isotonic solutions. The particles optionally include normal lung surfactants.

Aerosols may be formed of the particles in aqueous or nonaqueous (e.g., fluorocarbon propellant) suspension. Such particles include, for example, intramolecular aggregates of the therapeutic composition or derivatives thereof or liposomal or microcapsular-entrapped composition or derivatives thereof. The aerosols should be free of lung irritants, i.e., substances which cause acute bronchoconstriction, coughing, pulmonary edema or tissue destruction. However, nonirritating absorption-enhancing agents are suitable for use herein. Sonic nebulizers are preferably used in preparing aerosols. Sonic nebulizers minimize exposing the composition or derivatives thereof to shear, which can result in degradation of the composition.

Alternatively, the composition of the invention may be administered orally by delivery systems such as proteinoid encapsulation as described by Steiner, et al., U.S. Pat. No. 4,925,673, incorporated by reference herein. Typically, a therapeutically effective oral dose of a composition according to the invention is in the range from about 0.05 mg/kg body weight to about 50 mg/kg body weight per day a preferred effective dose is in the range from about 0.05 mg/kg body weight to about 5 mg/kg body weight per day.

Compositions according to the present invention may be administered systemically, rather than topically, by injection intramuscularly, subcutaneously, intrathecally or intraperitoneally or into vascular spaces, particularly into the joints, e.g., intraarticular injection at a dosage of greater than about 1 μg/cc joint fluid/day. The dose will be dependent upon the properties of the specific composition employed, e.g., its activity and biological half-life, the concentration of composition in the formulation, the site and rate of dosage, the clinical tolerance of the patient involved, the disease afflicting the patient and the like, as is well within the skill of the physician.

The compositions of the present invention may be administered in solution. The pH of the solution should be in the range of pH 5 to 9.5, preferably pH 6.5 to 7.5. The compositions thereof should be in a solution having a suitable pharmaceutically acceptable buffer such as phosphate, Tris(hydroxymethyl)aminomethane-HCl or citrate and the like. Buffer concentrations should be in the range of 1 to 100 mM. The composition solution may also contain a salt, such as sodium chloride or potassium chloride in a concentration of 50 to 150 mM. An effective amount of a stabilizing agent such as an albumin, a globulin, a gelatin, a protamine or a salt of protamine may also be included, and may be added to a solution containing composition or to the composition from which the solution is prepared.

Systemic administration of the composition may be made daily and is generally by intramuscular injection, although intravascular infusion is acceptable. Administration may also be intranasal or by other nonparenteral routes. Compositions of the present invention may also be administered via microspheres, liposomes or other microparticulate delivery systems placed in certain tissues including blood. Topical preparations are applied daily directly to the skin or mucosa and are then preferably occluded, i.e., protected by overlaying a bandage, polyolefin film or other barrier impermeable to the topical preparation.

The term “unit dosage” and its grammatical equivalents as used herein refer to physically discrete units suitable as unitary dosages for human patients and other warm blooded animals, each unit containing a predetermined effective and potentiating amount of at least one of the two active ingredients calculated to produce the desired therapeutic effect in association with the required physiologically tolerable carrier, e.g., a diluent or a vehicle. The specifications for the unit dosage forms of this invention are dictated by and are directly dependent on (a) the unique characteristics of the active ingredients and the particular therapeutic effect to be achieved, and (b) the limitations inherent in the art of compounding such as active ingredient for therapeutic use in humans and other animals. Examples of suitable unit dosage forms in accord with this invention are tablets, capsules, pills, powder packets, granules, wafers, and the like, segregated multiples of any of the foregoing, as well as liquid solutions, emulsions and suspensions. The amount of each active ingredient that is administered in vivo depends on the age and weight of the patient, the particular disease to be treated and its severity, the frequency of administration, and the route of administration.

In any treatment regimen, the therapeutic composition may be administered to a patient either singly or in a cocktail containing other therapeutic agents, compositions, or the like, including, but not limited to, immunosuppressive agents, tolerance-inducing agents, potentiators and side-effect relieving agents. Particularly preferred are immunosuppressive agents useful in suppressing allergic reactions of a host. Preferred immunosuppressive agents include prednisone, melphalain, prednisolone, DECADRON (Merck, Sharp & Dohme, West Point, Pa.), cyclophosphamide, cyclosporine, 6-mercaptopurine, methotrexate, azathioprine and i.v. gamma globulin or their combination. Preferred potentiators include monensin, ammonium chloride, perhexiline, verapamil, amantadine and chloroquine. All of these agents are administered in generally accepted efficacious dose ranges such as those disclosed in the Physician's Desk Reference, 41st Ed., Publisher Edward R. Barnhart, N.J. (1987). Patent Cooperation Treaty (PCT) patent application WO 89/069767 published on Aug. 10, 1989, which is incorporated by reference herein.

IV. Diagnostic Assays and Kits

In addition to the targeting of DNA encoding a cytotoxic agent to tumors in a cancer patient, the fusion polypeptide of the present invention also may be useful with certain types of tumor cells to detect the anatomic location of tumors. Such a determination can be useful for the subsequent planning of antitumor therapy in each particular patient. In particular, immunohistochemical pathologic diagnosis in tissue sections (e.g., biopsies), fluid samples (e.g., blood) or cytological preparations can be performed using the fusion polypeptides of the present invention.

In a preferred embodiment, detection is by the detection of a label bound to the fusion polypeptide. Means of labeling polypeptides are well known to those of skill in the art. Labels may be directly linked through a covalent bond or covalently through a linking molecule which typically bears reactive sites capable of forming covalent bonds with the label and the antibody respectively. a common approach is to label the polypeptide and the label with either avidin or streptavidin or biotin which, in turn, bind irreversibly with each other.

Suitable labels are well known to those of skill in the art. The term “label”, as used herein, refers to a composition detectable by spectroscopic, photochemical, biochemical, immunochemical, or chemical means. For example, useful labels include radioactive molecules such as ³²P, ¹⁴C, ¹²⁵I, ³H, and ³⁵S, fluorescent dyes such as fluorescein or rhodamine, electron-dense reagents, enzymes (as commonly used in an ELISA), luminescent enzymes such as luciferase and the like.

This invention also embraces kits for research or diagnostic purposes. A kit typically includes one or more containers containing the fusion polypeptide of the present invention, and optionally a second container containing a DNA molecule encoding a cytotoxie agent. Preferably the DNA sequence encoding the cytotoxic agent is provided in a plasmid suitable for transfection into and expression by a host cell. The plasmid may contain a promoter (often an inducible promoter) to regulate expression of the DNA in the host cell. The plasmid may also contain appropriate restriction sites to facilitate the insertion of other DNA sequences into the plasmid. The plasmids may also contain numerous other elements to facilitate cloning and expression of the encoded proteins. Such elements are well known to those of skill in the art and include, for example, selectable markers, initiation codons, termination codons, and the like.

The fusion polypeptides are themselves derivatized with a label or, alternatively, they may be bound with a secondary label to provide subsequent detection. As described above, such labels may include radiolabels, fluorescent labels, enzymatic labels, i.e., horseradish peroxidase (HRP), or the like. The kit may also contain appropriate secondary labels (e.g., a sheep antimouse-HRP, or the like). The kit may also contain various reagents to facilitate the binding of the fusion polypeptides, the removal of non-specific binding antibodies, and the detection of the bound labels. Such reagents are well known to those of skill in the art.

Methods for using the research and diagnostic kits described above are generally well known, and are generally provided in an instruction manual for use of the kit.

The invention will be further described by the following example.

### EXAMPLE 1

### Preparation of an Anti-CD38 scFv Construct

To determine whether a particular antibody (Ab) is useful in the therapeutic compositions of the invention, the binding of the antibody to normal cells (CD38 negative cells) and cells which express high levels of CD38 is determined. Specific binding can be determined by methods well known to the art including, but not limited to, flow cytometry. Cells for flow cytometry are washed in PBS+5% FCS and resuspended to approximately 10⁶/ml in the same buffer. Following the addition of Ab, cells are incubated at 4° C. for 30 to 60 minutes and washed several times in PBS+5% FCS. Bound Ab is visualized by incubating the cells with FITC-conjugates of anti-mouse Ig or anti-human Ig (Becton Dickinson and Sigrna) for 30 minutes at 4° C. After washing, reactions are analyzed by flow cytometry using a FACScan (Becton Dickinson). FITC-labeled anti-CD38 (clone HB7; Becton Dickinson) can be used as a positive control for CD38 expression. For inhibition studies, the first Ab is incubated with the cells for 45 minutes, and then the second Ab added without washing. After a further 45 minutes, the cells are washed and then visualized.

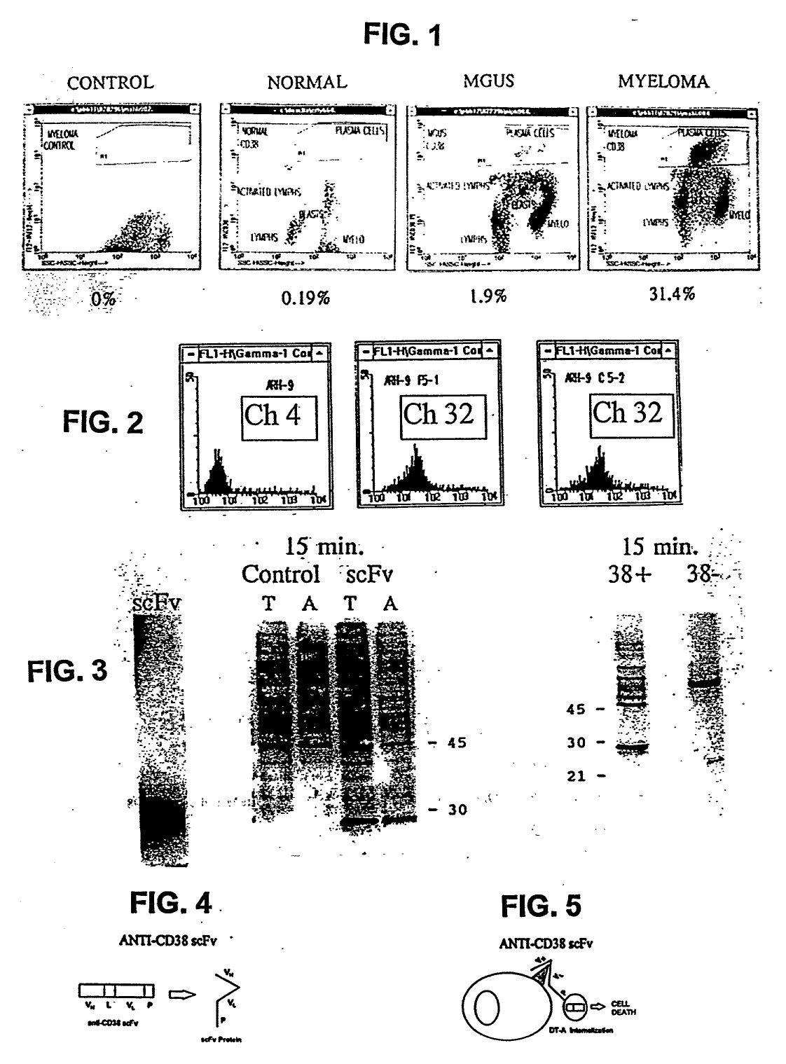

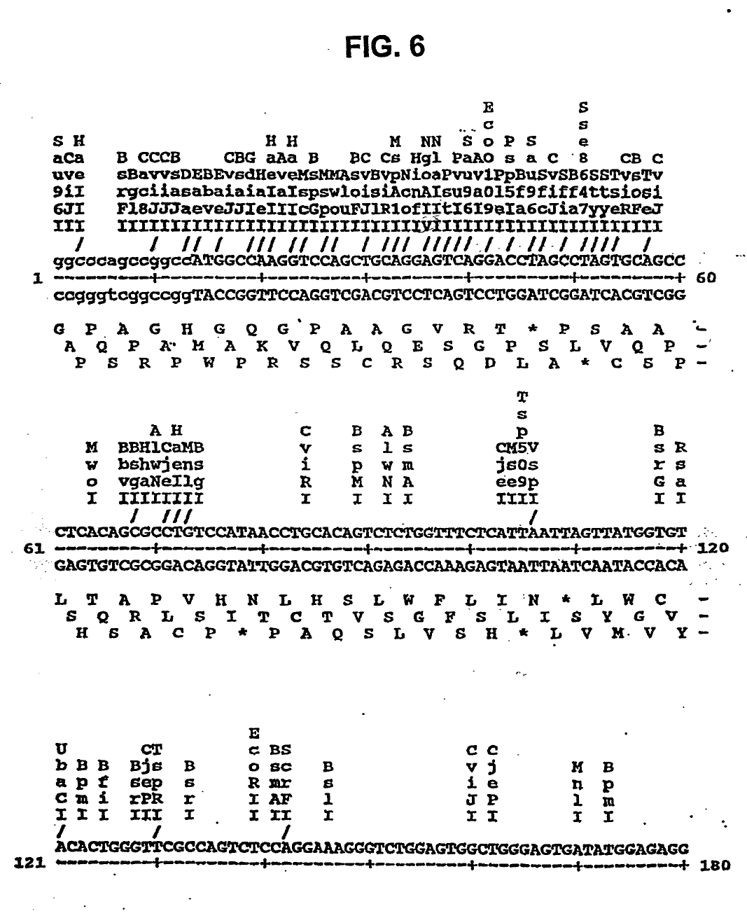

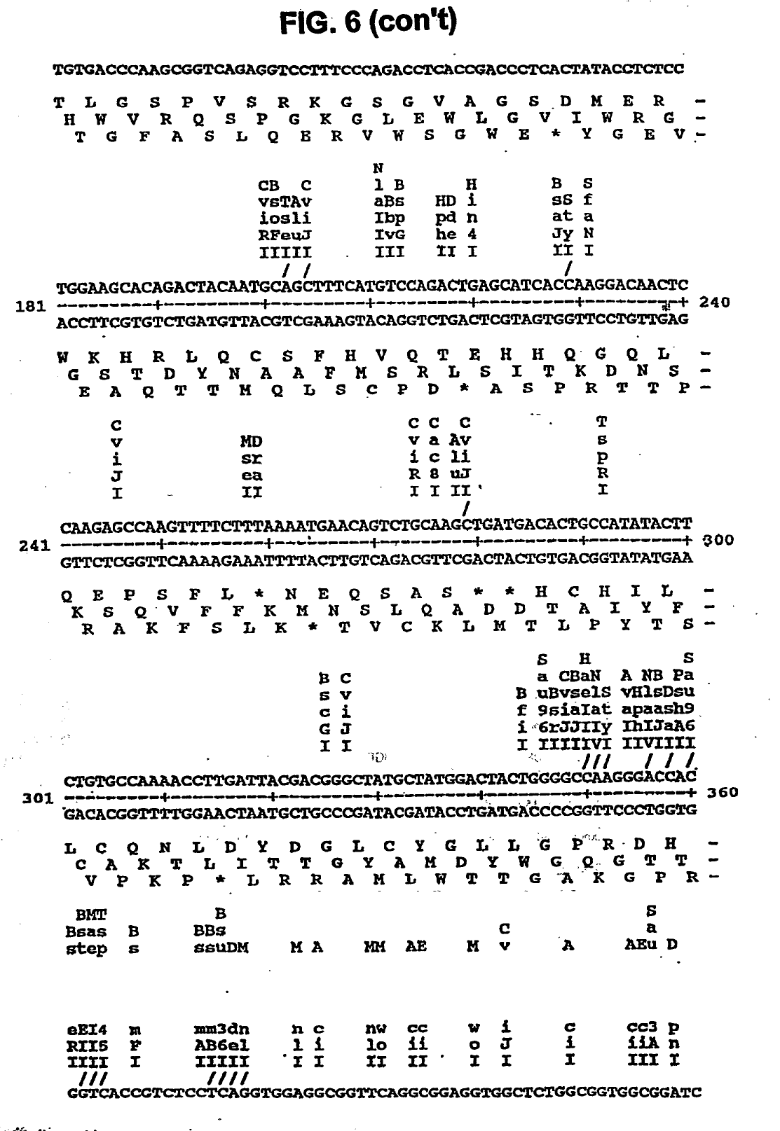

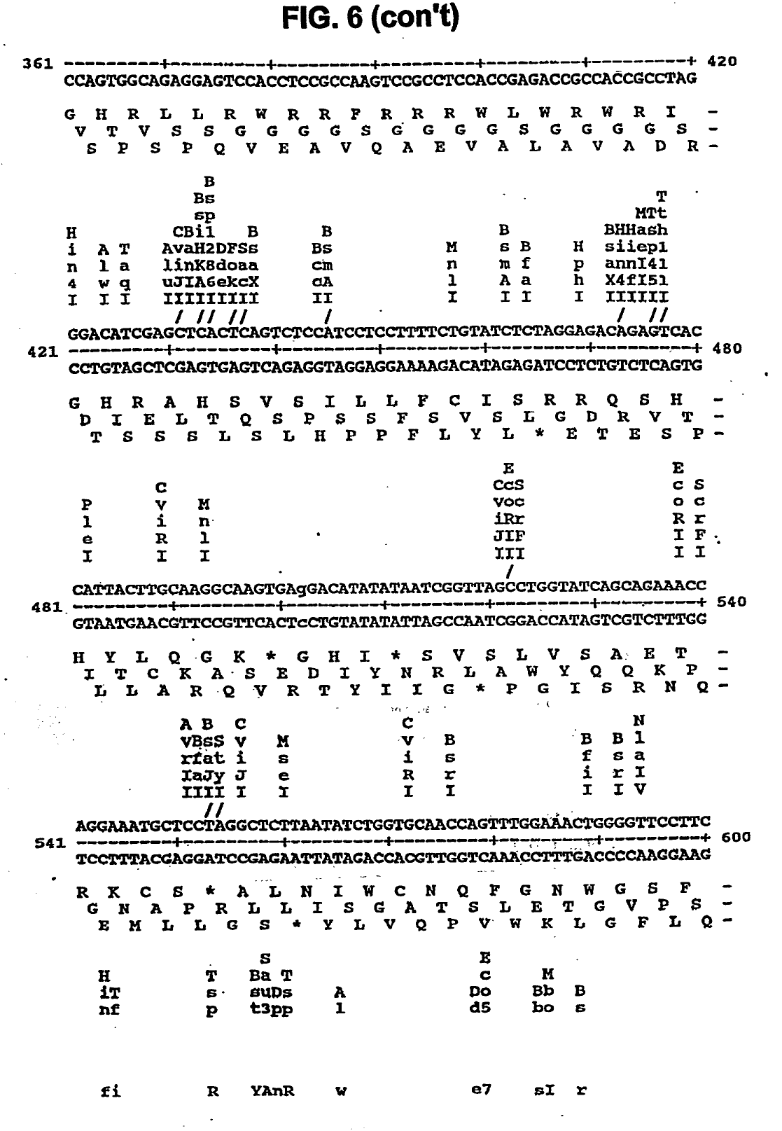

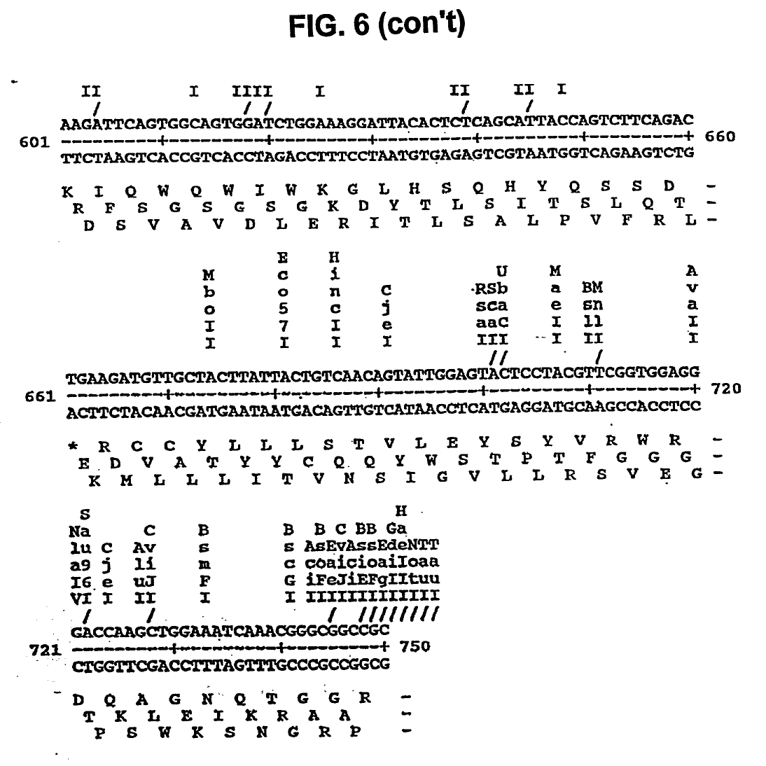

Bone marrow cells from a normal, MGUS (Kyle and Lust, Sem. Hemato., 26, 176 (1989)), and multiple myeloma patient were stained with an anti-CD38 antibody (FIG. 1). After setting similar gates for the plasma cell population on each sample, only 0.19% of normal cells expressed CD38 at levels comparable to clonal MGUS plasma cells (1.9%) or myeloma cells (31.4%).

Once a CD38-specific antibody has been identified, recombinant DNA technology can be employed to engineer portions of these antibodies. Genes encoding the variable heavy (V_{H}) and light (V_{L}) chain domains responsible for antigen recognition by immunoglobulin molecules can be modified genetically through the linkage of the carboxyl terminus of one variable region with the amino terminus of the other using nucleotides which encode a series of hydrophilic peptides. Compared to chemically generated Fabs, these scFv proteins retain the original antibody specificity and can have comparable stability with relatively minor changes in affinity. PCR technology is particularly useful to prepare scfvs because V_{H} and V_{L} regions can be easily cloned from hybridoma cells and combined with new functional domains. An scFv gene specific for CD38 was prepared from the isolated Ig heavy and light chain genes from an anti-CD38 hybridoma HB7 (ATCC Accession No. HB-136) by PCR.

V_{H} and V_{L} genes were then fused using PCR overlap extension techniques with appropriately constructed oligonucleotide primers to generate a 750 base pair V_{H}-linker-V_{L} scFv, using the expression module/recombinant phage antibody system according to the manufacturer's instructions (Pharmacia Biotech, Inc., Piscataway, N.J.). Optionally, at least a portion of the constant region of the Ig may also be introduced into the construct, which may result in desired properties, e.g., enhanced stability particularly in solution of the invention. All constructs are sequenced to exclude any clones having PCR-induced errors.

For protein expression and purification, vectors such as the pET vectors from Novagen (Madison, Wis.) may be employed. The pET 29 vector allows N-terminal fusion to a cleavable S-tag sequence for rapid assay and affinity purification. a multiple cloning site allows insertion of other DNAs of interest so that the resulting encoding the fusion polypeptide in the correct reading frame relative to the initiation codon.

Expression of the scFv construct generated a protein with a molecular weight of approximately 29 kDa. CD38 positive ARH cells were incubated with a control supernate versus expressed scFv polypeptide from two different scFv constructs, F5-1 and C5-2. Subsequently, each sample was incubated with an antibody to an E-tag (15 amino acid segments located in the scFv molecule), or, alternatively, an antibody to the introduced S-tag (see above), and a fluorescenated secondary antibody. The increase in the mean channel numbers (given in boxes) demonstrates the ability of the expressed scFv polypeptides from each of the two clones to bind to CD38 positive ARH cells. No differences in mean channel numbers were observed with CD38 negative cells (FIG. 2).

Internalization of the scFv polypeptide was detected by Western analysis. Western analysis of CD38 expressing 8226 myeloma cells previously incubated with the scFv polypeptide generated a 29 kDa band (FIG. 3). The 29 kDa band was not observed with CD38 negative cells. No differences were observed between total uptake (T) and uptake after an acid wash (a) indicating that the scFv polypeptide was endocytosed by the CD38+ tumor cells (FIG. 3). The finding that the scFv polypeptide is effectively internalized is significant because not all antibody molecules are endocytosed.

A DNA binding protein gene, e.g., a protamine gene (available from the American Tissue Culture Collection (ATCC) Accession No. 107507), is then added (fused) 3′ to the scFv construct (FIG. 4). Human protamine is a small basic DNA binding protein which serves to condense the entire human genomic DNA for packing into the restricted volume of a sperm head. The positive charges of the protamine strongly interact with the negative charges of the phosphate backbone of DNA resulting in a neutral and stable DNA-protamine complex. The scFv protamine molecule can interact with the toxin encoding plasmid DNA to form soluble protein-DNA complexes (FIG. 5).

The DNA binding ability of the scFv-protamine polypeptide to the diphtheria toxin a (DT-a) plasmid (complex formation) is assessed by a gel-mobility shift assay. The plasmid DNA is incubated with increasing amounts of the scFv-protamine polypeptide in 0.2 M NaCl at room temperature for 30 minutes and loaded on a 0.8% agarose gel for electrophoresis. Cytotoxicity of CD38 positive myeloma cells is measured by trypan blue staining after incubation of the cells in the anti-CD38 scFv protamine polypeptide alone, the DT-a plasmid alone, and the anti-CD38 protamine-DT-a complex. These complexes are internalized into antigen expressing cells, e.g., multiple myeloma cells, by receptor mediated endocytosis. Subsequent production of toxin protein from the plasmid DNA results in the selective killing of the cells.

Targeting of expression in B-cells may be achieved by linking a toxin gene with tissue-specific transcriptional regulatory elements such as immunoglobulin promoters and enhancers. For example, pTH73 DT-a (Maxwell et al., 1992) is a construct that contains an immunoglobulin heavy chain enhancer and an immunoglobulin kappa chain promoter that restricts the transcription of the DT-a gene to mature plasma cells. In addition, the DT-a gene lacks sequences for the domain of the naturally occurring diphtheria toxin required for cell binding and internalization. Therefore, the expressed protein which is released from lysed tumor cells is unable to affect bystander cells.

### References

EPA 0 105 360.

EPA 0 217 577.

WO83/03699.

WO96/16990.

U.S. Pat. No. 5,202,238.

U.S. Pat. No. 5,204,244.

U.S. Pat. No. 5,671,083.

Anderson, K., Cochran, M., Barut, B. Phenotypic and functional characterization of normal and malignant plasma cells. Eur. J. Haematol., 43, 19 (1990).

Batra et al., 1991, Mol. Cell Biol., 11:2200.

Chaudhary, V. K., Queen, C., Junghans, R. P., Waldmann, T. a., Fitzgerald, D. J., Pastan, I. a recombinant immunotoxin consisting of two antibody variable domains fused to Pseudomonas exotoxin. Nature, 339, 394-397 (1989).

Chen, S- Y, Zani, C., Khouri, Y., and Marasco, W. A., Design of a Genetic Immunotoxin to Eliminate Toxin Immunogenicity. Gene Therapy, 2, 116-123 (1995).

Ellis, J. H., Barber, K. A., Tutt, a., Hale, C., et al. Engineered anti-CD38 monoclonal antibodies for immunotherapy of multiple myeloma. J. Immunol., 155, 925-937 (1995).

Funaro, a., DeMonte, L. B., Dianzani, U., Forni, M., Malavasi, F. Human CD38 is associated to distinct molecules which mediate transmembrane signaling in different lineages. Eur. J. Immunol., 23, 2407-2411 (1993).

Glockshaber et al., 1990, Biochemistry, 29:1362.

Kaminski, M. S., Zasadny, K. R., Francis, I. R., Milik, A. W., Ross, C. W. et al. Radioimmunotherapy of B-cell lymphoma with [¹³¹]anti-B1(anti-CD20) antibody. N. Eng. J. Med., 322, 459-465 (1993).

Kohler and Milstein, 1975, Nature, 256:495.

Krawetz, S. a. et al. Chromosomal localization and structure of the human P1 protamine gene. Genomics, 5, 639-645 (1989).

Lord, J. M. Redirecting Nature's Toxins. Sem. Cell Biol., 2, 1-79 (1991).

Malavasi, F., Funaro, a., Roggero, S., Horenstein, a., Calosso, L., Mehta, K. Human CD38: a glycoprotein in search of a function. Immunol. Today, 15, 95-97 (1994).

Maxwell, I. H., Glode, L. M., Maxwell, F. Expression of diphtheria toxin a-chain in mature B-cells: a potential approach to therapy of B-lymphoid malignancy. Leukemia and Lymphoma, 7, 457-462 (1992).

Morrison et al., 1984, Proc. Natl. Acad. Sci. USA, 81:6851.

Neuberer et al., 1985, Nature, 314:268.

O'Hare et al., 1990, FEBS Lett., 273:200.

Palmiter, R. D., Behringer, R. R., Quaife, C. J., Maxwell, F., Maxwell, I. H., Brinster, R. L. Cell lineage ablation in transgenic mice by cell-specific expression of a toxin gene. Cell, 50, 435-443 (1987).

Pastan et al., 1986; Cell, 47:641.

Pastan and Fitzgerald, 1991, Science, 254:1173.

Riechmann et al., 1988, Nature, 332:323.

Siegall et al., 1988, Proc. Natl. Acad. Sci. USA, 85:9738.

Siegall, C. B., Chace, D., Mixan, B., Garrigues, U., Wan, H. et al. In vitro and in vivo characterization of BR96 sFv-PE40, J. Immunol., 152, 2377-2384 (1994).

Stevenson, F. K., Bell, a. J., Cusack, R., Hamblin, T. J., Slade, C. J., et al. Blood, 77, 1071-1079 (1991).

Thorpe et al., 1987, Cancer Res., 47:5924.

Vitetta et al., 1987, Science, 238:1098.

Ward et al., 1989, Nature, 341:544.

Westby et al., 1992, Bioconj. Chem., 3:375.

Winter and Milstein, 1991, Nature, 349:295.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A therapeutic composition, comprising:
a) a fusion polypeptide comprising a polypeptide which specifically binds CD38 or a portion thereof linked to a polypeptide which specifically binds DNA or a portion thereof; and
b) a DNA sequence encoding a cytotoxic agent which is operably linked to a cell- or tissue-specific transcriptional unit.

2. The composition of claim 1 wherein the polypeptide which specifically binds CD38 is an antibody.

3. The composition of claim 2 wherein the antibody is obtained from an antibody secreted by hybridoma HB7.

4. The composition of claim 1 wherein the polypeptide which specifically binds DNA is protamine.

5. The composition of claim 1 wherein the cytotoxic agent is diphtheria toxin A chain, a cell suicide protein, Pseudomonas exotoxin, or an enzyme or protein that activates a chemotherapeutic agent.

6. The composition of claim 1 wherein the transcription unit is specific for B cells.

7. The composition of claim 1 wherein the transcription unit is specific for T cells.

8. The composition of claim 1 wherein the transcription unit is specific for myeloid cells.

9. The composition of claim 2 wherein the antibody is a humanized antibody.

10. The composition of claim 1 further comprising a radioisotope linked to the fusion polypeptide.

11. The composition of claim 2 or 9 wherein the antibody is a scFv antibody.

12. An isolated and purified fusion polypeptide comprising at least a portion of a polypeptide that specifically binds CD38 and at least a portion of a polypeptide that specifically binds DNA.

13. A method to inhibit the growth of CD38+ cells, comprising contacting cells in vitro with an effective amount of the composition of claim 1.

14. An isolated and purified nucleic acid molecule comprising a nucleic acid segment encoding the fusion polypeptide of claim 12.

15. A method to inhibit or treat multiple myeloma, primary amyloidosis, monoclonal gammopathy, or acute myeloid leukemia, comprising:
administering to a mammal in need of said treatment an effective amount of the composition of claim 1.

16. A recombinant DNA molecule which encodes a single chain fusion polypeptide, wherein the recombinant DNA molecule comprises:
a) a DNA sequence that encodes the Fv region of a light chain of an antibody specific for CD38 and the Fv region of a heavy chain of an antibody specific for CD38, wherein the fusion protein binds to CD38⁺ cells; and
b) a DNA sequence that encodes a polypeptide that specifically binds DNA.

17. A recombinantly produced single chain fusion polypeptide comprising:
a) the Fv region of the light and the heavy chain of a CD38 specific antibody; and
b) a DNA binding polypeptide, wherein the Fv region and the DNA binding polypeptide are recombinantly fused to form a single chain polypeptide that specifically binds CD38⁺ cells.

18. A pharmaceutical composition comprising a recombinantly produced single chain fusion polypeptide in a concentration sufficient to inhibit tumor cell growth, together with a pharmaceutically acceptable carrier wherein the fusion polypeptide comprises:
a) a single chain Fv region of an antibody, wherein the Fv region comprises the V_{H} and V_{L} regions of the antibody; and
b) a DNA binding polypeptide, wherein the Fv region and the DNA binding polypeptide are recombinantly fused to form a single molecule that specifically binds CD38⁺ cells.
